(19) United States
(12) Patent Application Publication
(10) Pub. No.: US 2005/0148974 A1
(43) Pub. Date: Jul. 7, 2005

(54) Disposable absorbent garment having a folded panel for improved fit and exudate containment

(76) Inventors: Paul J. Datta, Appleton, WI (US); Mary Jo Meyer, Neenah, WI (US)

Correspondence Address: BRINKS HOFER GILSON & LIONE, P.O. BOX 10395, CHICAGO, IL, 60610, US

(21) Appl. No.: 10/747,853

(22) Filed: Dec. 29, 2003

(51) Int. Cl. A61F 13/15; A61F 13/20

(52) U.S. Cl. 604/385.01; 604/385.03; 604/387

(57) ABSTRACT

A disposable absorbent article garment having a body panel, an extension fold extending from the rear waist edge of the body panel and an absorbent insert and defining an opening. At least one garment closing fastener is connected to the body side surface of the extension fold and a portion of the body panel. In another aspect, the extension fold is gathered a first amount and a portion of the body panel is gathered a second amount, wherein the first amount of gathering is greater than the second amount of gathering. A method of making a disposable absorbent garment includes folding a body panel web to form an extension fold, connecting an absorbent insert to the body panel and then applying at least one garment closing fastener to the extension fold and the body panel.

### BACKGROUND

The present invention relates generally to disposable absorbent garments, and in particular, to a disposable absorbent garment having an adjustable extension fold and fastener system that provides an improved fit and containment for bodily exudates, and also to the method for making the disposable absorbent garment.

Disposable absorbent garments can be configured in many different forms. For example, disposable absorbent garments can be configured as a pant-type product, a pull-on garment, or as a diaper-type product that is drawn up between the legs and fastened about the waist with various fastening systems. In some configurations, the disposable absorbent garment has a waist feature allowing the user to adjust the overall length of the garment. In some configurations, the disposable absorbent garment is configured with a folded down waist feature disposed in the rear waist region. The waist feature extends laterally outward and includes a fastening system attached thereto, as disclosed for example in U.S. Pat. No. 5,989,236. In one system, the folded down waist feature folds over the garment side of the chassis, but does not form a pocket to collect any bodily exudates. In an other system, the disposable absorbent garment does not have any fasteners that secure the portion of the chassis underlying the waist feature to the front portion of the chassis, such that the unsecured portion of the chassis can move away from the front portion and permit leakage.

Therefore, the need remains for an improved absorbent garment that has a flexible waist feature capable of allowing the garment to be adjusted to fit a variety of users but securely retains body exudates without leakage. Further, there is a need for an improved adjustable waist feature that conforms to the body of the user during use without interferences from the absorbent insert.

### SUMMARY

Briefly stated, in one embodiment, a disposable article includes a body chassis comprising a body panel and an extension fold folded over at least a first portion of the body panel. The extension fold and the first portion of the body panel define a folded edge along a waist edge of the body chassis. The extension fold extends longitudinally from the folded edge in an overlying relationship with the first portion of the body panel. The extension fold terminates in a free edge, wherein at least a portion of the free edge is not connected to the body panel such that the free edge of the extension fold and the first portion of the body panel form an opening therebetween. The body panel has at least a second portion with a body side surface extending longitudinally from the free edge of the extension fold such that the body side surface of the second portion does not underlie the extension fold. An absorbent insert is connected to the body panel. At least one garment closing fastener member is connected to the body side surface of the extension fold and the body side surface of the second portion.

In another embodiment, a disposable article includes a first, front body panel and a second, rear body panel. The second rear body panel comprises an extension fold folded over at least a first portion of the second rear body panel. The extension fold and the first portion define a folded edge along a waist edge of the rear body panel. The extension fold extends longitudinally from the folded edge in an overlying relationship with the first portion of the rear body panel. The second rear body panel has at least a second portion with a body side surface extending longitudinally from the free edge of the extension fold such that the body side surface of the second portion does not underlie the extension fold. An absorbent insert is disposed between the first, front body panel and the second, rear body panel. At least one garment closing fastener member is attached to the body side surface of the extension fold and the body side surface of the second portion of the rear body panel.

In another aspect, a method of making a disposable article includes folding a body panel web and thereby forming an extension fold folded over at least a first portion of the body panel web. The method further includes connecting an absorbent insert to the body panel web. In addition, the method includes applying at least one garment closure fastener to the body side surface of the extension fold and the body side surface of a second portion of the body panel web extending away from the extension fold.

In another embodiment, the extension fold is gathered a first amount and the first portion of the body panel is gathered a second amount, wherein the first amount of gathering is greater than the second amount of gathering.

The various embodiments described herein provide significant advantages over other disposable undergarments and methods of making the disposable undergarment thereof. For example, the rear body panel has an extension fold that can be folded to conform to the body of the user, and thereby provide a snug fit to the body of the user. In addition, the garment closing fastener member connecting the rear body panel to the front body panel secures the extension fold and the second portion to the front body panel, thereby providing a secure fit when used. In addition, the difference in the gathering of the extension fold and first portion of the body panel allows expansion of the pocket to receive exudates.

The foregoing paragraphs have been provided by way of general introduction, and are not intended to limit the scope of the following claims. The presently preferred embodiments, together with further advantages, will be best understood by reference to the following detailed description taken in conjunction with the accompanying drawings.

### BRIEF DESCRIPTION OF THE DRAWINGS

Many of the features and dimensions portrayed in the drawings, and in particular the presentation of layer thicknesses and the like, and the spacing therebetween, have been somewhat exaggerated for the sake of illustration and clarity.

FIG. 1 is a plan view of a first embodiment of a disposable absorbent article taken from the body side thereof.

FIG. 2 is a plan view of a second embodiment of a disposable absorbent article taken from the body side thereof.

FIG. 3 is a plan view of a third embodiment of a disposable absorbent article taken from the body side thereof.

FIG. 4 is a plan view of a portion of a fourth embodiment of a disposable absorbent article taken from the body side thereof.

FIG. 5 is a detailed plan view of the fourth embodiment of a disposable absorbent article shown in FIG. 4 with the extension fold in an unfolded configuration.

FIG. 6 is a cross-sectional view of the disposable absorbent article taken along 6-6 in FIG. 2.

FIG. 7 is a cross-sectional view of the disposable absorbent article taken along 7-7 in FIG. 1.

FIG. 8 is a cross-sectional view of the disposable absorbent article taken along 8-8 in FIG. 1.

FIG. 9 is a cross-sectional view of a portion of the disposable absorbent article taken along line 9-9 of FIG. 5.

FIG. 10 is plan view of an alternative embodiment of a disposable absorbent article with an extension fold in an unfolded configuration.

### DETAILED DESCRIPTION OF THE PRESENTLY PREFERRED EMBODIMENTS

Referring to FIG. 1, it should be understood that the term “longitudinal,” 500 as used herein, means of or relating to length or the lengthwise direction. The term “laterally,” 502 as used herein, means situated on, directed toward or running from side to side. The term “first direction” generally refers to a path, line or course rather than a vector, and includes and applies equally to opposite orientations along the path, line or course, including for example and without limitation movement along a path, line or course in both directions (as indicated by the bidirectional arrows associated with the longitudinal and lateral directions 500, 502). Likewise, the term “second direction” generally refers to a path, line or course rather than a vector (not orientation dependent), and includes for example and without limitation movement along a path, line or course in both directions. In one example, the first direction is defined by and refers to one of the longitudinal and lateral directions, while the second direction refers to the other of the longitudinal and lateral directions.

The term “bodyside” should not be interpreted to mean in contact with the body of the user, but rather simply means the side that would face toward the body of the user, regardless of whether an undergarment is actually being worn by the user and regardless of whether there are or may be intervening layers between the disposable absorbent article and the body of the user. Likewise, the term “garment side” should not be interpreted to mean in contact with the garments of the user, but rather simply means the side that faces away from the body of the user, and therefore toward any outer garments that may be worn by the user, regardless of whether the undergarment is actually being worn by a user, regardless of whether any such outer garments are actually worn and regardless of whether there may be intervening layers between the component and any outer garment.

The phrases “releasably attached,” “releasably connected,” or “releasably secured,” and variations thereof, refers to two or more elements being connected or connectable such that the elements tend to remain connected absent a separation force applied to one, both or all of the elements, and where the elements are capable of being separated upon the application of a separation force. The required separation force is typically beyond that encountered while wearing the absorbent disposable article.

The terms “connected,” “attached,” and “secured,” and variations thereof, broadly covers two or more items being directly connected one to the other, or by way of one or more intervening members or components.

Referring to FIGS. 1, 7 & 8, a disposable article 2 has a body chassis member 8 that includes a first front body panel 4 and a second, rear body panel 6. The terms “body chassis member” and “body panel” refer to the portion(s) of the disposable article, whether made of one or more layers or substrates or of one or more pieces or components, that is/are fitted circumferentially around the body of the user, for example about the waist region of the user, and/or one or more of the user's lower back, buttock, hips, crotch and abdomen.

The first and second body panels each have an inner, body side surface 10 and an outer, garment side surface 12. The first front body panel 4 has a first terminal edge 14 formed along the crotch region of the garment and second terminal edge 20 formed along the waist region of the disposable article. In one embodiment, the first and second terminal edges 14, 20 are linear but can assume other shapes. Likewise, the second, rear body panel 6 has a first terminal edge 22 formed along the crotch region of the disposable article and a second terminal edge 18 that is formed along the waist region of the disposable article. In one embodiment, the first and second terminal edges 18, 22 are linear but can assume other shapes.

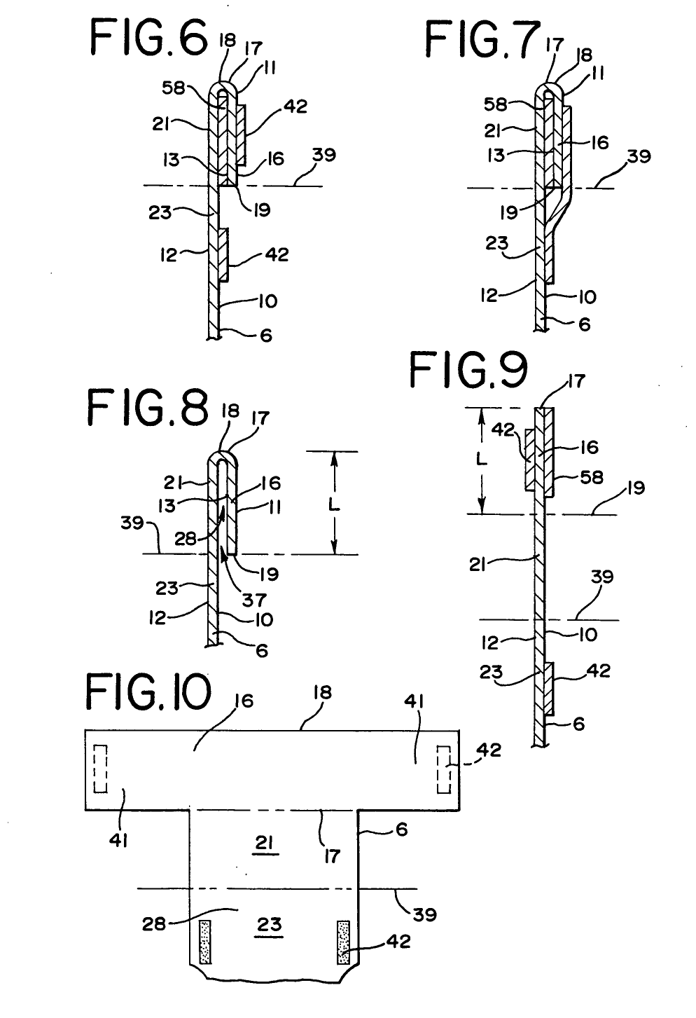

As shown in FIGS. 6, 7 and 8, the rear body panel 6 has an extension fold 16 folded along a folded edge 17 and extending longitudinally from the terminal waist edge 18 and terminating at a free edge 19. The extension fold 16 has an inner, body side surface 11, and an outer garment side 13, corresponding respectively to the garment side surface 12 and body side surface 10 of the body panel when the extension fold is unfolded as shown in FIG. 9.

Each of the first and second body panels has an outboard side edge 30, 32 formed along the outer periphery of the opposite side portions of the first and second body panels. In one embodiment, the side edges 30, 32 are the same length, although it should be understood that the outboard side edges of the front and rear body panels could have different lengths relative to each other. In one embodiment, the contoured edges 24, 26 connect the terminal edges 14, 22 with the side edges 30, 32 respectively and define a portion of a leg opening 34. In yet another embodiment, the terminal edges 14, 22 are linear and extend across the entire lateral width of the disposable article without any tapered edges. In one embodiment, shown in FIG. 10, the extension fold 16 has ear portions 41 that extend outwardly from side edges of a first portion 21 of the rear body panel 6.

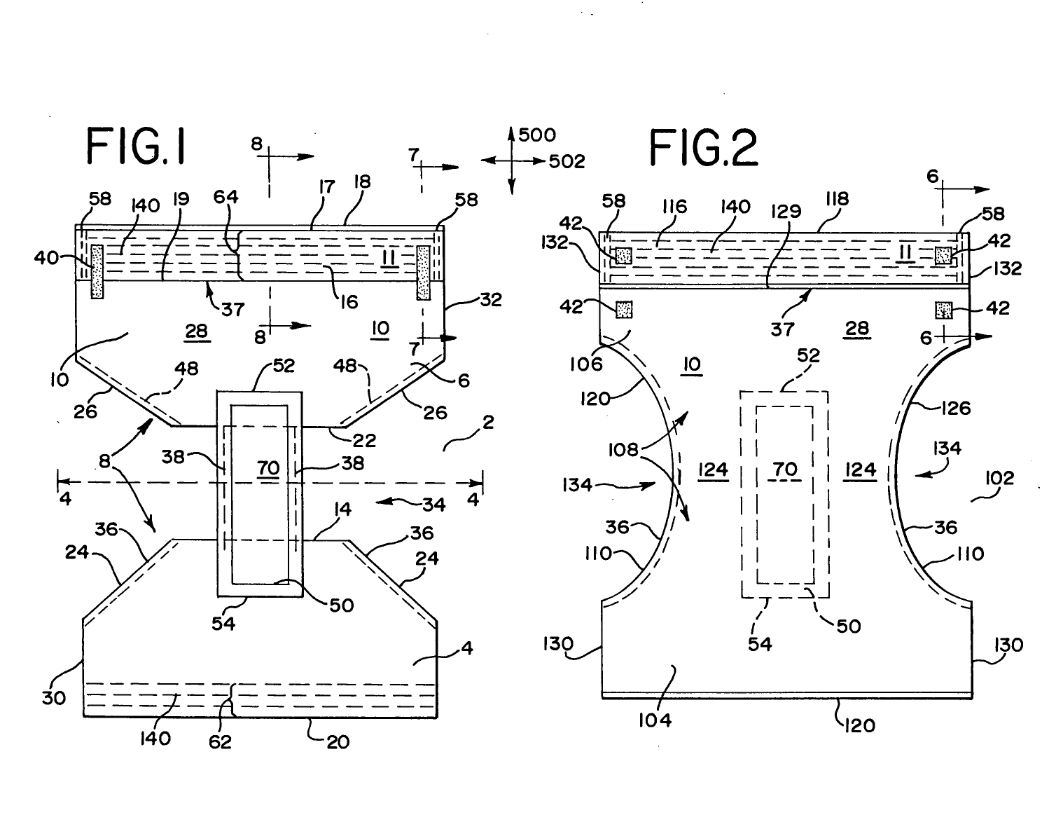

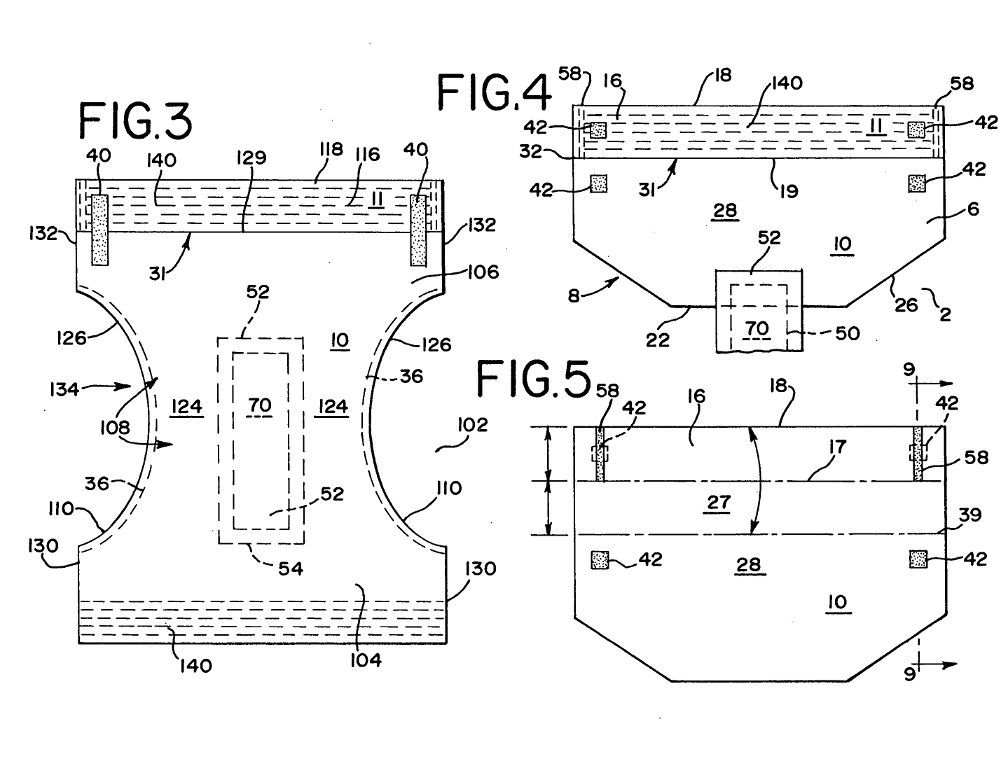

In an alternative embodiment, shown in FIGS. 2 and 3, the body chassis member 108, including an outer cover, extends substantially the entire length of the disposable article and integrally defines the first, front body panel 104 and the second, rear body panel 106, as well as a crotch portion 124 extending therebetween. In this embodiment, with the front and rear body panels 104, 106 integrally formed with a crotch portion 124, the body panels have curved edges 110, 126 that form the front and rear portions of the leg opening 134, the side edges 130, 132 and opposite terminal edges 120, 118. In addition, the rear body panel has an extension fold 116 that extends longitudinally from the terminal edge 118 and terminates in a free edge 129.

In one embodiment, shown for example in FIGS. 1-4, one or more elastic elements 140 are secured to at least one of the first and second body panels 4, 6, 104, 106. In one embodiment shown, a plurality of elastic elements 140 are spaced longitudinally across substantially the entire portion 62, 64 of the front and rear body panels, although they may be spaced across a lesser length. Exemplary elastic materials are described in U.S. Pat. No. 4,977,011, to Smith issued Dec. 11, 1990 entitled “Disposable Elastic Structure” and U.S. Pat. No. 5,209,801 to Smith, issued May 11, 1993, entitled “Method of Forming a Disposable Elastic Structure,” both of which are hereby incorporated herein by reference.

In one embodiment, shown in FIG. 1, one or more crotch elastic elements 38 are sandwiched between a top sheet and back sheet of the crotch portion 124 along the side edges thereof. In other embodiments, the crotch elastic elements 38 may be applied to the top sheet, the back sheet, and/or as separate bands. The crotch elastic elements 38 can extend the entire longitudinal length of the absorbent insert, or along only a portion thereof.

In one embodiment, a plurality (meaning two or more) of elastic elements 140 are disposed along the extension fold 16, 116 and along at least a portion 21 of the body panel 6, 106. In one embodiment, there are more elastic elements 140 disposed along the extension fold 16, 116 than along the underlying portion 21 of the rear body panel 6, 106. In one embodiment, there are at least about ten elastic elements 140 disposed along each of the extension fold 16, 116 and the portion 21 of the rear body panel 6, 106. In one embodiment, the extension fold 16, 116 is gathered a first amount and the first portion 21 is gathered a second amount, with the first amount of gathering being greater than the second amount of gathering. In this way, the elastic elements 140 allow the extension fold 16, 116 to gather more than the portion 21 of the rear body panel, which will ensure a better fit, and create a pocket 28 between the extension fold 6, 16 and the first portion 21.

For example, in one embodiment, the pocket 28 is enhanced by at least a portion of the extension fold 16, 116 being gathered more than the portions 21, 23 of the body panel as shown in FIG. 4. The un-gathered body panel is thereby allowed to expand outwardly from the extension fold so as to thereby create a pocket for waste material. The gathered extension fold 16, 116 is securely closed to the body of the user creating a gasket to protect against leakage of bodily exudates. Elastic members 140 are added to both the extension fold 16 and the unfolded back waist portion 21. The differences in the amount of gathering can be effected in several different ways, including providing more elastic elements 140 along the extension fold 16, 116 than in the underlying portion 21, by altering the spacing of the elastic elements 140 secured to the extension fold different from the first portion, by elongating the elastic elements 140 secured to the extension fold 16, 116 a greater amount than the elastic elements secured to the first portion 21, by altering the material use as elastic elements 140 and/or by elongating the extension fold 16, 116 a greater amount that the first portion prior to attaching the elastic elements 140. Different elastic materials are defined by the denier, size or chemistry that could be different to produce different retractive forces to affect the gathering. In one embodiment, the elastic elements 140 are applied to only the extension fold 16, 116. If constructed as a separate piece, the extension fold 16, 116 can be attached to the portion 21, for example and without limitation by bonding (adhesive, ultrasonic, pressure, etc.), stitching, mechanical fasteners and the like.

The tension difference, or difference in the amount of gathering in the extension fold 16, 116 and the first portion 21, can be measured by the difference in the retraction between the lateral width of the extension fold 16, 116 and the lateral width of portion 21 of the rear body panel 6, 106. To make this measurement, a sample is made by cutting one of the front and rear body panels along a border 39 defined by the free edge 19, 119 such that the sample is formed by the extension fold 16, 116 and the portion 21. The portion 21 and the extension fold 16, 116 are then opened relative to each other and laid flat. The extension fold 16, 116 is then separated from the first portion 21 by cutting along the fold edge 18, 118 so that you have two pieces of material the same size. The two pieces are stretched in the lateral direction 502 to smooth any wrinkles that may have formed while processing the disposable article. Next, steel rulers, e.g., No R590-24 rulers from Westcott, are positioned on the pieces to keep wrinkles from reforming. The extension fold piece and the first portion piece, and in particular any elastics forming a part thereof, are allowed to retract for five minutes. The relaxed lateral widths of the extension fold 16, 116 and the underlying portion 21 are then measured. It is important that an equal length of the extension fold 16, 116 and portion 21 be allowed to be retracted. Finally, the difference between the measured lateral width of the relaxed extension fold 16, 116 from the measured lateral width of the relaxed underlying body panel portion 21 defines the retracted width difference.

In one embodiment, the initial (pre-relaxed) length of the sample (or the lateral width of the extension fold and underlying portion) was about 11.0 inches. In a desired embodiment, the extension fold retracts a greater amount than the underlying body panel. In various embodiments, the retracted width difference for an 11.0 inch sample is between about 0.10 inches and 6.0 inches. More desirably, the difference is between about 0.25 inches and 5.5 inches and most desirably, the difference is between about 0.5 inches and 5.0 inches. It should be understood that samples having other lengths may also exhibit retracted width differences in the ranges provided herein.

In addition, it should be understood that in various embodiments, the retracted width difference can be defined in percentages, which is not dependent on the initial sample length. In various embodiments, the retracted width different is between about 0.90% and about 75% of the initial un-relaxed length of the sample (extension fold and underlying portion). In other embodiments, the retracted width difference is less than about 60% of the initial length of the sample. In other embodiments, the retracted width difference is greater than about 1% of the initial length of the sample. In other embodiments, the retracted width difference is between about 1% and about 55% of the initial length of the sample, alternatively between about 2% and about 40% of the initial length of the sample.

In various embodiments, the extension fold 16, 116, can be made of different materials and/or as a separate piece from the first portion 21. Referring to FIGS. 5-9, the extension fold 16, 116 is inwardly folded over a first portion 21 of the body facing surface 10 to create the pocket 28 for waist containment. The free edge 19, 129 forms the opening 31 for the pocket 28 with the body side surface 10 as shown in FIG. 8. The longitudinal length (L) of the extension fold 16, 116 (measured from waist to crotch) can be adjusted from a first length to a second length simply by longitudinally moving the fold line 17. The length (L) is adjustable between about 0.5 and 12 inches, and more desirably is between about 2 and 10 inches, and even more desirably between about 4 and 8 inches. A second portion 23 of the body side surface remains uncovered by the extension fold and extends longitudinally away from the free edge 19, 129, or the border 39 between the first portion and the second portion defined by the free edge.

In another embodiment, as shown in FIG. 1, a permanent continuous side edge seal or a refastenable extension fold fastener 58 is used to seal the side edges of the waste pocket 28, and in particular to connect the garment side surface 13 of the extension fold 16 to the bodyside surface 10 of the first portion 21 of the body panel 6. The permanent continuous side edge seal prevents leakage from the edges of the garment. In one embodiment the extension fold refastenable fasteners can be adjusted for users with different requirements for product length. Of course, it should be understood that the extension fold is not necessarily secured to the first portion 21.

As used herein the term extension fold fastener refers to fasteners securing the extension fold to a portion of the underlying body panel, and the term garment closing fasteners refers to fasteners securing the rear and front body panels to each other, for example around the body of the user.

Referring to FIG. 1, as shown in one embodiment, the elastic elements 140 can also be attached along front body panel waist portion 62 adjacent to the first terminal edge 20 and along the contoured edges 24, 26, defining in part a leg opening 34. It should be understood, that in various embodiments, one or more separate waist bands, with or without elastic elements, can be secured to one or both of the rear and front body panels, preferably along or near the upper terminal edges 18, 20, 118, 120 thereof.

Likewise, one or more separate leg elastic elements 36 can be secured to one or both of the rear and front body panels along the edge 24, 26 of the body panel and/or along the edge of the crotch portion forming and defining the leg openings 34. One or more separate leg elastic elements 36 can be secured to one or both of the rear and front body panels along the curved edge 110 of the body chassis 108 and/or along the edge of the crotch portion forming and partially defining the leg openings 134. Leg elastic elements 36, may be also positioned in the crotch region of the absorbent insert 50. The leg elastic elements 36 may be elastic to gasket the leg and prevent leakage in use. The leg elastic elements 36 may be linear or curved in part or in total. The leg elastic elements 36 follow the contoured edges 24, 26 of the rear and front body panels 4, 6 more clearly define the leg openings 34. The leg elastic elements 36 may overlap to completely encircle the leg openings 34, or may have gaps between the leg elastic elements 36 for comfort. Alternatively, one or both of the body panels can be formed without any elastic elements. The various elastic elements 140 can be formed from rubber or other elastomeric materials. One suitable material is a Lycra® elastic material. For example, the various elastic elements can be formed of Lycra® XA Spandex 540, 740 or 940 decitex T-127 or T-128 elastics available from E.I. duPont De Nemours and Company, having an office in Wilmington, Del.

In one embodiment, the leg opening 34, 134 is not symmetrical. The leg opening 34, 134 is set forward so that the ratio of the side edge of the unfolded back waist portion is at least 1.2 times the length of the front waist side edge.

Referring to FIGS. 1-4, each body panel 4, 6, 104, 106 is preferably formed as a composite, or laminate material, otherwise referred to as substrates or laminates, with an elastic core sandwiched therebetween. In one embodiment, the elastic core is made of an elastomeric film or non-woven elastic or stretchable material including for example block copolymers of polystyrene, polyisoprene or polybutadiene, copolymers of ethylene, natural rubbers, urethanes, kratons, and co-extrusions/blends of the aforementioned material. The elastic core can be formed as a membrane or from a plurality of elastic strands, as described above. In one embodiment, two or more layers are bonded to the elastic core, and/or each other, with various adhesives, such as hot melt, or by other techniques, including for example and without limitation ultrasonic bonding and heat and/or pressure sealing. In one embodiment, the two layers are made of a non-woven material such as a spunbond material, a bonded carded material or other known materials. In this way, the body panels are made of a stretchable/elastic material.

It should be understood that the body panels 4, 6, 104, 106 can be made of a single layer or substrate of non-woven material, a bi-layer substrate made of non-woven materials without an elastic core, or more than two layers or substrates. Of course, it should be understood that other knitted or woven fabrics, non-woven fabrics, elastomeric materials, polymer films, laminates and the like can be used to form one or more of the body panel layers. The term “non-woven” web or material, as used herein, means a web having a structure of individual fibers or filaments that are interlaid, but not in an identifiable manner and without the aid of textile weaving or knitting, as in a knitted or woven fabric.

In one embodiment, the front and rear body panel material can be secured to the elastic core, such as an elastomeric layer or elastic strands or ribbons, which have been elongated and retracted, such that the material is gathered when the elastic element(s) are relaxed. Alternatively, the material can be gathered and laminated to non-elongated elastic elements. In one embodiment, the body panel 4, 6, 104, 106 includes a gathered elastic laminate made from non-woven base sheets bonded with elongated elastic elements sandwiched therebetween.

In various embodiments, the body panel material may be substantially permeable to air or substantially impermeable to air. The body panel material also may be substantially liquid-permeable or substantially liquid-impermeable. In one embodiment, the back sheet of the body panel material includes at least one layer which is a moisture proof barrier material, but desirably comprises a material which is configured to be substantially impermeable to liquids. The barrier material protects the user in containing waste and keeps waste contained in the disposal process. For example, a typical back sheet can be manufactured from a thin plastic film, or other flexible, substantially liquid-impermeable material. The back sheet prevents the exudates contained in absorbent insert from wetting articles, such as bed sheets and over garments.

Alternative constructions of the back sheet may comprise a woven or non-woven fibrous web layer which has been totally or partially constructed or treated to impart the desired levels of liquid impermeability to selected regions that are adjacent or proximate to the absorbent insert. For example, the back sheet may include a gas-permeable, non-woven fabric layer laminated to an appointed facing surface of a polymer film layer that may or may not be gas-permeable. In particular arrangements, a substantially liquid impermeable, vapor permeable back sheet layer may be a composite material which includes a vapor permeable film layer adhesively laminated to a spunbond lawyer. One suitable vapor permeable film layer can be obtained from redegar Film Products, under the tradename EXAIRE. The film layer can include 48-60 weight percent (wt %) linear low density polyethylene and 38-50 wt % calcium carbonate particulates that may be uniformly dispersed and extruded into the film layer. The stretch film layer can have a thickness of about 0.7 mil (about 0.018 mm) and a basis weight of 16-22 grams per square meter (g/m2). The spunbond layer can adhesively laminated to the film layer, and can have a basis weight of about 27 g/m2. The spunbond layer can made using conventional spunbond technology, and can include filaments of polypropylene having a fiber denier of 1.5-3 dpf. The vapor-permeable film layer may be adhered to the spunbond layer using a pressure sensitive, hot melt adhesive at an add-on rate of abut 1.6 g/m, and the adhesive can be deposited in the form of a pattern of adhesive swirls or a random fine fiber spray. As mentioned, the back sheet may include a micro-porous, “breathable” material which permits gases, such as water vapor, to escape from the absorbent body while substantially preventing liquid exudates from passing through the back sheet. For example, the breathable back sheet may be composed of a microporous polymer film or a non-woven fabric that has been coated or otherwise modified to impart a desired level of liquid impermeability. Another example of a suitable microporous film can be a PMP-1 material, which is available from Mitsuit Toatsu Chemicals, Inc., a company having offices in Tokyo, Japan; or an XKO-8044 polyoefin film available from 3 M Company of Minneapolis, Minn.

In particular arrangements, the body panel material may be substantially nonelastomeric. In other aspects, the body panels can include an elastomeric material that is elastomerically stretchable at least along one or both of the lateral article widths and/or one or more of the longitudinal article length. Examples of such elastomeric composite materials can include a continuous filament stretch bonded laminate (CFSBL), a vertical filament laminate (VFL), neck-bonded-laminate (NBL), a stretch-bonded-laminate (SBL), a necked-stretch bonded laminate (NSBL) or a necked-thermal laminate, or the like, as well as combinations thereof. Exemplary CFSBL, NBL, SBL, and NSBL materials are described in U.S. Pat. Nos. 5,226,992, 4,981,747, 4,965,122, 5,336,545, 5,385,775, 5,414,470, 4,720,415, 4,789,699, 4,781,966, 4,657,802, 4,652,487, 4,655,760, 5,116,662 and 5,114,781, and 6,323,389, all of which are hereby incorporated herein by reference. Exemplary VFL materials are described in U.S. Provisional Patent Application Ser. No. 60/204,307, filed May 15, 2000 and entitled “Method and Apparatus for Producing Laminated Articles,” and PCT application WO 01/88245 A2, both assigned to Kimberly-Clark Worldwide, Inc., the Assignee of the present application, with the entire disclosures of both being hereby incorporated herein by reference. Such laminates can provide an improved combination of cloth-like feel and elastomeric stretchability. The body panels 4, 6, 104, 106 can be composed of materials that are elastic or elastomeric and exhibit biaxial stretch characteristics or lateral/longitudinal stretch characteristics, or which are extensible composites. Additional waist and leg elastic elements can be added to, but are not necessarily required by, the body panels 4, 6, 104, 106.

In one embodiment, the body panel material is extensible but not elasticized. For example, the body panel can be made of a film or non-woven that is attached, by way of adhesives or thermal bonding, to an extensible non-woven material. Alternatively, the body panel can be made of a low modulus film such as Ethylene Methyl acrylate (EMA).

It should be understood that, in one embodiment, the entirety of the body panels 4, 6, 104, 106, the extension fold, 16, 116 and the crotch portion 124 of chassis 108, can be elasticized, such that the entirety of each of the body panels can elongate and conform to the body of the user without any substantial spacing between the body panel and the user's body, and without the attendant bulkiness of a non-elasticized material.

In one embodiment, the body panels are breathable, cloth-like, multi-directional non-woven laminates with stretch or extensible properties. In one embodiment, the non-woven layers are pre-necked, for example between about 10% and about 80%, in the longitudinal direction, which provides extensibility in the longitudinal direction with minimal force.

In one embodiment, the body chassis member 8, 108 are made of non-woven laminates of two layers of longitudinally extensible 0.60 osy polypropylene spunbond material with elongated strands of Lycra® elastic sandwiched between the spunbond layers and thereafter adhesively bonded. In particular, the body panel material is necked in the cross direction. As used herein, the term “necked,” and variations thereof, refers to any material that has been constricted in at least one dimension by applying a tensioning force in a direction that is perpendicular to the desired direction of neck-down. Processes that may be used to constrict a material in such a manner include, for example and without limitation, drawing processes. The elastics are then elongated in the direction 502 and secured to the body panel material. The elastics are then allowed to retract so as to gather the necked spunbond material in the direction 502 thereby creating an elastically gathered non-woven body panel with longitudinal extensibility. The term “gather,” and variations thereof, as used herein means puckered, or contracted into folds or wrinkles, which should be understood as including micro-pleats.

In this way, the rear body panel 6, 106 and extension fold 16, 116 can be elongated in both the direction 500 and the direction 502 to conform to the body of the user when the disposable article is applied thereto. In particular, as the user pulls the garment up over their hips, the non-woven laminate body panels stretch in the lateral direction while the leg regions of the front and rear body panels conform to the crotch and body lines of the user. At the same time, the body panel material extends in the longitudinal direction to conform to the buttocks and stomach of the user. The extension fold 16, 116 is folded inwardly to provide a secure fit to the user. The extension fold 16, 116 may be secured to the body facing surface of the rear body panel using the extension fold fastener 58, whether relatively permanent or adjustable. It should be understood that the fastening systems includes use of adhesives, sealants, or equivalent products that can be used to secure the extension fold, 16, 116 to the body side surface of the rear body panel 6, 106. The extensibility of the body panels 4, 6, 104, 106 follows the natural curvature of user's body to provide conformance thereto. As the body panel extends in the longitudinal machine direction 500, the spacing between the laterally extending elastic elements, incorporated in one embodiment, will increase.

In one embodiment, the body chassis non-woven material is preferably substantially hydrophobic, which may optionally be treated with a surfactant or otherwise processed to impart a desired level of wettability and hydrophilicity. In one particular embodiment of the invention, the body panel is a non-woven, wire-weave spunbond polypropylene fabric composed of about 1.6 denier fibers formed into a web having a basis weight of about 0.6 osy. One suitable non-woven material is the Corinth 0.60 osy, 1.6 dpf wireweave, nonwettable Metallocene (EXXON ACHIEVE 2854 PP) spunbond material manufactured by Kimberly-Clark Corporation, the assignee of the present application.

In one embodiment, the crotch portion of the various undergarments connecting the front and rear body panels 4, 6, 104, 106, whether integrally formed or separately connected with an absorbent insert, can be folded such that the side edges 30, 32, 130, 132 of the front and rear body panels 4, 6, 104, 106 are aligned, whereinafter they can be fixedly secured at a seam to form the leg opening 34, 134. The seam can be formed by bonding, sewing or otherwise attaching the side edges. Alternatively, the product can remain “open,” wherein the body panels are releasably secured with one or more garment closure fasteners as explained below.

In one embodiment, extension fold fasteners 58 are releasably secured between the extension fold 16 and the first portion 21 of the body panel to allow the length of the undergarment to be adjusted for a customized fit. For example, in one embodiment, extension fold fasteners 58 are preferably attached to the garment side surface 12 of the extension fold 16, 116 and are releasably secured to the body side surface 10 of the first portion 21. Alternatively, the extension fold fasteners 58 are attached to the first portion and releasably engage the extension fold 16. In one embodiment, a two piece extension fold fastening system can be used where at least one element is secured to the extension fold 16 and at least one mating element is secured to the first portion 21 in a manner such that the elements engage one another to produce a side edge 17 for the pocket when the extension fold is folded onto the body side surface 10 of the first portion 21.

In one embodiment, shown in FIGS. 1, 3 and 7, a plurality (shown as two) of garment closing fastener members 40 are secured to the body side surface 11 of the extension fold and the body side surface 10 of the second portion 23. The term “plurality” as used herein means two or more. The garment closing fastener members 40 may extend across the free edge 19 and help secure the extension fold 16 to the second portion 23 of the body panel 6, thereby also functioning as an extension fold fastener. In this embodiment, the garment closing fastener members are preferably applied after the extension fold 16 is folded over the first portion 21.

In an alternative embodiment, shown in FIGS. 2, 4, 5, 6, 9 and 10, the garment closing fastener members 42 are discrete elements and do not extend across the free edge 19. In this embodiment, the garment closing fastener members 42 can be applied either after or before the extension fold 16 is folded over the first portion 21. For example, as shown in FIGS. 5, 9 and 10, the garment closure fastener members 42 are applied to the extension fold 16 before it is folded along line 17.

In either embodiment, the garment closure fasteners members 40, 42 engage the front body panel 4, 104. In one embodiment, the garment fastener members 40, 42 are configured as a hook and loop fastener. The garment closure fastener members 40, 42 can releasably engage a landing material (not shown), or the body panel material itself. Of course, it should be understood that the garment fastener members can be any structure configured to secure one member to another, and can include without limitation adhesives, buttons, pins, snaps, electrostatic fields, magnetic fields, mechanical fasteners and other known structures.

Referring to the embodiments of FIGS. 1-4, an absorbent insert 50 is formed as a separate subassembly connected to a bodyside surface of the body chassis member 8 (whether formed as a single integral member having a front and rear body panel and a crotch portion as shown in FIGS. 2 and 3, or as discrete and spaced apart front and rear body panel members as shown in FIGS. 1). In either embodiment, the absorbent insert 50 has a first and a second opposed terminal end edge 52, 54. In the embodiment of FIGS. 1 and 4, the absorbent insert 50 bridges the gap between the terminal edges 14, 22 of the body panels 4, 6 and is connected respectively to those body panels. In an alternate embodiment, as shown in FIGS. 2 and 3, the absorbent insert 50 is integrated with the body chassis 108.

In one embodiment, the absorbent insert 50 includes a substantially liquid permeable top sheet, or liner, and a substantially liquid impermeable back sheet. A retention portion 70 is disposed or sandwiched between the top sheet and the back sheet, which are connected. The top sheet, back sheet and other components of the absorbent insert can be joined for example with adhesive bonds, sonic bonds, thermal bonds, pressure bonds, pinning, stitching or any other attachment techniques known in the art, as well as combinations thereof. For example, a uniform continuous layer of adhesive, a patterned layer of adhesive, a sprayed pattern of adhesive or any array of lines, swirls or spots of construction bonds may be used to join the top sheet and back sheet, or any of the other components described herein.

Referring to FIGS. 1 and 4, the garment side of the end regions of the absorbent insert 50, and in particular, the outer, garment side surface of the back sheet, are connected to the body side surface of the first and second body panels 4, 6 at attachment locations. It should be understood that in an alternative embodiment, the absorbent insert, for example the body side thereof, can be connected to the garment side surface of the first and second body panels. It should be understood that the absorbent insert 50 can be secured using any of the methods of attachment described above, including for example various adhesives, stitching or other bonding methods. The absorbent insert can be secured to the body panels with any configuration of attachment lines, swirls, patterns, spots, etc., or can be a full and continuous attachment therebetween. In one embodiment, a portion of the absorbent insert 50 underlies the extension fold 16, 116.

Additional layers, including for example, a liquid acquisition and distribution layer also referred to as a surge or transfer layer, are also preferably incorporated into the absorbent insert. In one embodiment, the transfer layer does not run the entire length of the absorbent insert and is shorter than the retention portion.

The retention portion 70 is made of an absorbent material, which can be any material that tends to swell or expand as it absorbs exudates, including various liquids and/or fluids excreted or exuded by the user. For example, the absorbent material can be made of airformed, airlaid and/or wetlaid composites of fibers and high absorbency materials, referred to as superabsorbents. Superabsorbents typically are made of polyacrylic acids, such as FAVOR 880 available from Stockhausen, Inc. of Greensboro, N.C. The fibers can be fluff pulp materials, such as Alliance CR-1654, or any combination of crosslinked pulps, hardwood, softwood, and synthetic fibers. Airlaid and wetlaid structures typically include binding agents, which are used to stabilize the structure. In addition, various foams, absorbent films, and superabsorbent fabrics can be used as an absorbent material. Various acceptable absorbent materials are disclosed in U.S. Pat. Nos. 5,147,343 for Absorbent Products Containing Hydrogels With Ability To Swell Against Pressure, 5,601,542 for Absorbent Composite, and 5,651,862 for Wet Formed Absorbent Composite, all of which are hereby incorporated herein by reference. Furthermore, the proportion of high-absorbency particles can range from about 0 to about 100%, and the proportion of fibrous material from about 0 to about 100%. Additionally, high absorbency fibers can be used such as Oasis type 121 and type 122 superabsorbent fibers available from Technical Absorbent Ltd., Grimsby, Lincolnshire, United Kingdom.

The retention portion 70 preferably can be made of a single or dual layer of absorbent material. In one embodiment, the retention portion 70 has an hour-glass shape with enlarged end regions. Alternatively, the retention portion 70 is substantially rectangular. The retention portion 70 can include a folded or multi-layered configuration. The retention portion 70 can have a length substantially equal to, or slightly shorter than, the length of the absorbent insert. The absorbent insert 50 can include one or more barrier layers attached to the absorbent material. In one embodiment, an upper tissue substrate is disposed adjacent the retention portion 70. Alternatively, a lower tissue substrate can be disposed adjacent an opposite side of the retention portion, or the tissue can completely envelope the retention position 70.

A method of manufacturing a disposable article 2 includes moving the body chassis member 8 in the lateral direction 502 and attaching elastic elements 140. An extension fold fastener 58 is applied to the body side surface 10 of the first portion 21 or the garment side surface, after folding, of the extension fold 16, 116. In one embodiment, extension fold fasteners 58 would be applied to both the body side surface 10 of portion 21 and the garment side surface 12 of the extension fold. The extension fold fasteners 58 are applied near or adjacent to the side edges 32 of the second rear body panel 6. Two extension fold fasteners 58 can be applied to each disposable article 2. A slip cut module as disclosed in U.S. Pat. No. 6,599,384 issued to Milner et al., entitled “A Method of Separating and Placing Discrete Elements,” the entire disclosure of which is hereby incorporated herein by reference, may be used to incrementally apply the fasteners.

The free edge 19 of the extension fold 16 is folded using a folding board as described in U.S. Pat. No. 1,046,325 issued to Normand entitled “Paper-Folding Apparatus.,” the entire disclosure of which is hereby incorporated herein by reference. The fold line 17 is set using a simple set of nip rollers over the fold line. The nip rollers may also extend over the free edge 19 and engage the extension fold fasteners 58. An absorbent insert 50 is applied and connected to the chassis member 8 before or after the operation. At least one pair of garment closure fasteners 40, 42 is applied to the rear body panel 6 in a fashion such that at least part of the garment closure fastener 40, 42 is attached to the extension fold on the body facing surface 10 and to the portion 23 on the body side surface 11. The crotch portion is folded such that the front body panel 4 is disposed adjacent to the rear body panel 6, with side edges 30 being aligned with side edges 31. A lateral crotch fold line is generated by matching up the side edge to form leg openings. Nip rollers are used to engage the garment fastener 40, 42. In an alternate embodiment the garment closing fastener 40, 42 may be replaced with a permanent bond using adhesive, pressure and/or heat seal or sonic bonding that is applied after generating the crotch fold. It should be understood that the front body panel can also have an extension fold as herein described.

Although the present invention has been described with reference to preferred embodiments, those skilled in the art will recognize that changes may be made in form and detail without departing from the spirit and scope of the invention. As such, it is intended that the foregoing detailed description be regarded as illustrative rather than limiting and that it is the appended claims, including all equivalents thereof, which are intended to define the scope of the invention.

1. An absorbent article comprising:
a body chassis comprising a body panel and an extension fold folded over at least a first portion of said body panel, said extension fold and said first portion defining a folded edge along a waist edge of said body chassis, said extension fold extending longitudinally from said folded edge in an overlying relationship with said first portion of said body panel, said extension fold having a body side surface and a garment side surface facing a body side surface of said first portion, and wherein said extension fold terminates in a free edge, wherein at least a portion of said free edge is not connected to said body panel such that said free edge of said extension fold and said first portion of said body panel form an opening therebetween, and wherein said body panel has at least a second portion with a body side surface extending longitudinally from said free edge of said extension fold such that said body side surface of said second portion does not underlie said extension fold;
an absorbent insert connected to said body panel; and
at least one garment closing fastener member connected to said body side surface of said extension fold and said body side surface of said second portion of said body panel.

2. The absorbent article of claim 1 wherein said extension fold is foldable between at least a first configuration and a second configuration, wherein said extension fold has a first length in said first configuration and said extension fold has a second length in said second configuration, wherein said second length is greater than said first length.

3. The absorbent article of claim 1 wherein at least a portion of said garment side surface of said extension fold is secured to said first portion of said body panel.

4. The absorbent article of claim 3 wherein said at least said portion of said garment side surface of said extension fold is releasably secured to said first portion of said body panel.

5. The absorbent article of claim 3 wherein said at least said portion of said garment side surface of said extension fold is secured to said first portion of said body panel with an adhesive.

6. The absorbent article of claim 1 wherein said at least one garment closing fastener comprises a continuous element extending from said body side surface of said extension fold to said second portion of said body panel across said free edge of said extension fold.

7. The absorbent article of claim 1 wherein said at least one garment closing fastener comprises at least one first garment closing fastener member connected to said body side surface of said extension fold and at least one second garment closing fastener member connected to said body side surface of said second portion of said body panel, wherein said at least one first garment closing fastener member is separate from said at least one second garment closing fastener member.

8. The absorbent article of claim 7 wherein said second garment closing fastener member extends under said extension fold and connects a garment side surface of said extension fold and a body side surface of said first portion of said body panel, wherein said second garment closing fastener comprises a extension fold fastening portion connecting said garment side surface of said extension fold and said body side surface of said first portion.

9. The absorbent article of claim 1 wherein said body panel comprises a rear body panel having a terminal crotch edge, and further comprising a front body panel having a terminal crotch edge spaced from said terminal crotch edge of said rear body panel, wherein said terminal crotch edges of said front and rear body panels form a gap therebetween, wherein said absorbent insert is connected to said front and rear body panels and bridges said gap.

10. A method of making an absorbent article comprising:
providing a body panel web;
folding said body panel web and thereby forming an extension fold folded over at least a first portion of said body panel web, said extension fold having a body side surface and a garment side surface facing a body side surface of said first portion, wherein said extension fold terminates in a free edge, wherein at least a portion of said free edge is not connected to said body panel web such that said free edge of said extension fold and said first portion of said body panel web form an opening therebetween, and wherein said body panel web has at least a second portion with a body side surface extending longitudinally from said free edge of said extension fold such that said body side surface of said second portion does not underlie said extension fold;
connecting an absorbent insert to said body panel web; and
applying at least one garment closing fastener to said body side surface of said extension fold and said body side surface of said second portion of said body panel web.

11. The method of claim 10 further comprising applying adhesive between a garment side surface of said extension fold and said body side surface of said first portion, wherein said extension fold and said first portion form a waste containment pocket.

12. The method of claim 11 wherein said applying said adhesive comprises applying said adhesive longitudinally between said folded edge and said free edge in at least a pair of spaced apart and substantially continuous patterns so as to define said waste containment pocket.

13. The method of claim 10 further comprising securing at least a portion of said garment side surface of said extension fold to said first portion of said body panel web.

14. The method of claim 13 wherein said securing said at least said portion of said garment side surface of said extension fold to said first portion of said body panel web comprises releasably securing said at least said portion of said garment side surface of said extension fold to said first portion of said body panel web.

15. The method of claim 13 wherein said securing said at least said portion of said garment side surface of said extension fold to said first portion of said body panel comprises securing said at least said portion of said garment side surface of said extension fold to said first portion of said body panel with an adhesive.

16. The method of claim 10 wherein said at least one garment closing fastener comprises a continuous element extending from said body side surface of said extension fold to said second portion of said body panel across said free edge of said extension fold.

17. The method of claim 10 wherein said fastener comprises at least one first garment closing fastener member connected to said body side surface of said extension fold and at least one second garment closing fastener member connected to said body side surface of said second portion of said body panel web, wherein said at least one first garment closing fastener member is separate from said at least one second garment closing fastener member and wherein said at least one first and second garment closing fastener members do not cross said free edge.

18. An absorbent article comprising:
a body chassis comprising a body panel and an extension fold folded over at least a first portion of said body panel, said extension fold and said first portion defining a folded edge along a waist edge of said body chassis, said extension fold extending longitudinally from said folded edge in an overlying relationship with said first portion of said body panel, said extension fold having a body side surface and a garment side surface facing a body side surface of said first portion, and wherein said extension fold terminates in a free edge, wherein at least a portion of said free edge is not connected to said body panel such that said free edge of said extension fold and said first portion of said body panel form an opening therebetween, and wherein said body panel has at least a second portion with a body side surface extending longitudinally from said free edge of said extension fold such that said body side surface of said second portion does not underlie said extension fold;
wherein said extension fold is gathered a first amount and said first portion of said body panel is gathered a second amount, wherein said first amount of gathering is greater than said second amount of gathering.

19. The absorbent article of claim 18 wherein said extension fold is retractable a first distance from a first position to a second position, and wherein said first portion is retractable a second distance from said first position to a third position, wherein said first distance is greater than said second distance.

20. The absorbent article of claim 19 wherein the difference between said first and second distances is between about 0.10 inches and about 6.00 inches.

21. The absorbent article of claim 20 wherein the difference between said first and second distances is between about 0.50 inches and about 5.00 inches.

22. The absorbent article of claim 18 wherein said extension fold comprises a first number of elastic elements and said first portion comprises a second number of elastic elements, wherein said first number is greater than said second number.

23. The absorbent article of claim 18 wherein the difference between said first and second distances is between about 1% and 60% of an initial length of said extension fold and said first portion when in said first position.

24. A method of making an absorbent article comprising:
moving a body panel web in a first direction;
attaching a plurality of elastic elements to said body panel web;
folding said body panel web and thereby forming an extension fold folded over at least a first portion of said body panel web, wherein a majority of said plurality of elastic elements are positioned on said extension fold, said extension fold having a body side surface and a garment side surface facing a body side surface of said first portion, wherein said extension fold terminates in a free edge, wherein at least a portion of said free edge is not connected to said body panel web such that said free edge of said extension fold and said first portion of said body panel web form an opening therebetween, and wherein said body panel web has at least a second portion with a body side surface extending longitudinally from said free edge of said extension fold such that said body side surface of said second portion does not underlie said extension fold.

25. The method of claim 24 further comprising:
connecting an absorbent insert to said body panel web; and
applying at least one garment closing fastener to said body side surface of said extension fold and said body side surface of said second portion of said body panel web.

26. The method of claim 24 further comprising connecting said extension fold to said first portion of said body panel web.
